(19) United States
(12) Patent Application Publication
(10) Pub. No.: US 2016/0051220 A1
(43) Pub. Date: Feb. 25, 2016

(54) ULTRASOUND DIAGNOSIS APPARATUS AND ULTRASOUND DIAGNOSIS METHOD

(71) Applicant: SAMSUNG MEDISON CO., LTD., Hongcheon-gun (KR)

(72) Inventors: Kwang-hee LEE, Hongcheon-gun (KR); Jae-heung YOO, Seoul (KR)

(73) Assignee: SAMSUNG MEDISON CO., LTD., Hongcheon-gun (KR)

(21) Appl. No.: 14/830,184

(22) Filed: Aug. 19, 2015

(30) Foreign Application Priority Data
Aug. 19, 2014 (KR) 10-2014-0107761

(51) Int. Cl. A61B 8/08; A61B 8/00

(52) U.S. Cl. CPC A61B 8/085; A61B 8/463; A61B 8/466; A61B 8/483; A61B 8/488; A61B 8/5207

(57) ABSTRACT

Provided is an ultrasound diagnosis apparatus. The ultrasound diagnosis apparatus includes an image acquiring unit configured to acquire a three-dimensional (3D) ultrasound image of a partial region of an object, a region determining unit configured to determine a bile duct region and a tumor candidate region in the 3D ultrasound image; a resection pattern acquiring unit configured to acquire a resection pattern of the bile duct region by comparing a shape of the tumor candidate region and the bile duct region with a predetermined pattern; and a display unit configured to display on a screen a resection pattern image including the resection pattern of the bile duct region.

### RELATED APPLICATION

This application claims the benefit of Korean Patent Application No. 10-2014-0107761, filed on Aug. 19, 2014, in the Korean Intellectual Property Office, the disclosure of which is incorporated herein in its entirety by reference.

### BACKGROUND

1. Field

One or more exemplary embodiments relate to ultrasound diagnosis apparatuses and ultrasound diagnosis methods.

2. Description of the Related Art

Ultrasound diagnosis apparatuses transmit ultrasound signals generated by transducers of a probe to an object and receive echo signals reflected from the object, thereby obtaining at least one image of an internal part of the object. In particular, ultrasound diagnosis apparatuses are used for medical purposes including observation of the interior of an object, detection of foreign substances, and diagnosis of damages to the object. Such ultrasound diagnosis apparatuses have high stability, display images in real time, and are safe due to the lack of radioactive exposure, compared to X-ray apparatuses. Therefore, ultrasound imaging apparatuses are widely used together with other image diagnosis apparatuses.

### SUMMARY

One or more exemplary embodiments include ultrasound diagnosis apparatuses and ultrasound diagnosis methods.

Additional aspects will be set forth in part in the description which follows and, in part, will be apparent from the description, or may be learned by practice of the presented exemplary embodiments.

According to one or more exemplary embodiments, an ultrasound diagnosis apparatus includes: an image acquiring unit configured to acquire a three-dimensional (3D) ultrasound image of a partial region of an object; a region determining unit configured to determine a bile duct region and a tumor candidate region in the 3D ultrasound image; a resection pattern acquiring unit configured to acquire a resection pattern of the bile duct region by comparing a shape of the tumor candidate region and the bile duct region with a predetermined pattern; and a display unit configured to display on a screen a resection pattern image including the resection pattern of the bile duct region.

The region determining unit may determine gallbladder candidate regions by comparing regions in the 3D ultrasound image which have a pixel brightness value less than or equal to a threshold value with a predetermined shape, and determine the largest of the gallbladder candidate regions as a gallbladder region by comparing sizes of the gallbladder candidate regions.

The region determining unit may determine bile duct candidate regions by comparing regions which are adjacent to the gallbladder region and have a pixel brightness value less than or equal to a threshold value with a predetermined shape, and determine the largest of the bile duct candidate regions as the bile duct region by comparing sizes of the bile duct candidate regions.

The region determining unit may determine the tumor candidate region on the basis of at least one of a Doppler signal of the bile duct region and a shape of the bile duct region.

When the tumor candidate region displayed on the screen is modified by an external input, the resection pattern acquiring unit may modify the resection pattern on the basis of the modified tumor candidate region and the display unit may display the modified resection pattern on the screen.

The display unit may display on the screen an ultrasound image including at least one of the gallbladder region, the bile duct region, and the tumor candidate region.

The display unit may display an auxiliary image on the screen, wherein the auxiliary image is a 3D-rendered image of the ultrasound image.

The display unit may display at least one of the resection pattern image, the ultrasound image, and the auxiliary image on the screen.

The display unit may display a blood vessel, the bile duct region, and the gallbladder region of the auxiliary image in different colors.

The display unit may display the tumor candidate region of the auxiliary image in a different color in the bile duct region.

The display unit may display the tumor candidate region according to at least one type of indicator.

According to one or more exemplary embodiments, an ultrasound diagnosis method includes: acquiring a 3D ultrasound image of a partial region of an object; determining a bile duct region and a tumor candidate region in the 3D ultrasound image; acquiring a resection pattern of the bile duct region by comparing a shape of the tumor candidate region and the bile duct region with a predetermined pattern; and displaying on a screen a resection pattern image including the resection pattern of the bile duct region.

### BRIEF DESCRIPTION OF THE DRAWINGS

These and/or other aspects will become apparent and more readily appreciated from the following description of exemplary embodiments, taken in conjunction with the accompanying drawings in which:

FIG. 1 is a block diagram of an ultrasound diagnosis apparatus according to an exemplary embodiment;

FIG. 2 illustrates types of bile duct tumors;

FIG. 3 illustrates computerized tomography (CT) images, rendered images, and resection pattern images of a bile duct and a gallbladder;

FIG. 4 is a block diagram of an ultrasound diagnosis apparatus according to some exemplary embodiments;

FIG. 5 is a flow diagram of an ultrasound diagnosis method according to some exemplary embodiments;

FIG. 6 illustrates a tumor display screen of an ultrasound diagnosis apparatus according to some exemplary embodiments;

FIG. 7 illustrates a screen displaying a resection pattern image based on a determined tumor candidate region according to some exemplary embodiments; and

FIG. 8 is a diagram illustrating an example of displaying an ultrasound image, an auxiliary image, and a resection pattern image on one screen according to some exemplary embodiments.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description. As used herein, expressions such as “at least one of,” when preceding a list of elements, modify the entire list of elements and do not modify the individual elements of the list.

The terms used in the present specification are general terms currently widely used in the art in consideration of functions regarding the inventive concept, but the terms may vary according to the intention of those of ordinary skill in the art, precedents, or new technology in the art. Also, some terms may be arbitrarily selected by the applicant, and in this case, the meaning of the selected terms will be described in detail in the detailed description of the present specification. Thus, the terms used herein have to be defined based on the meaning of the terms together with the description throughout the present specification.

Throughout the specification, it will also be understood that when a component “includes” an element, unless there is another opposite description thereto, it should be understood that the component does not exclude another element and may further include another element. In addition, terms such as “ . . . unit”, “ . . . module”, or the like refer to units that perform at least one function or operation, and the units may be implemented as hardware or software or as a combination of hardware and software.

Throughout the specification, an “ultrasound image” refers to an image of an object, which is acquired by using ultrasound waves. Furthermore, an “object” may be a human, an animal, or a part of a human or animal. For example, the object may be an organ (e.g., a liver, a heart, a womb, a brain, a breast, or an abdomen), a blood vessel, or a combination thereof. Also, the object may be a phantom. The phantom may mean a material having a density, an effective atomic number, and a volume that are approximately the same as those of an organism.

Throughout the specification, a “user” may be, but is not limited to, a medical expert, for example, a medical doctor, a nurse, a medical laboratory technologist, or a medical imaging expert, or a technician who repairs medical apparatuses.

Hereinafter, exemplary embodiments will be described in detail with reference to the drawings.

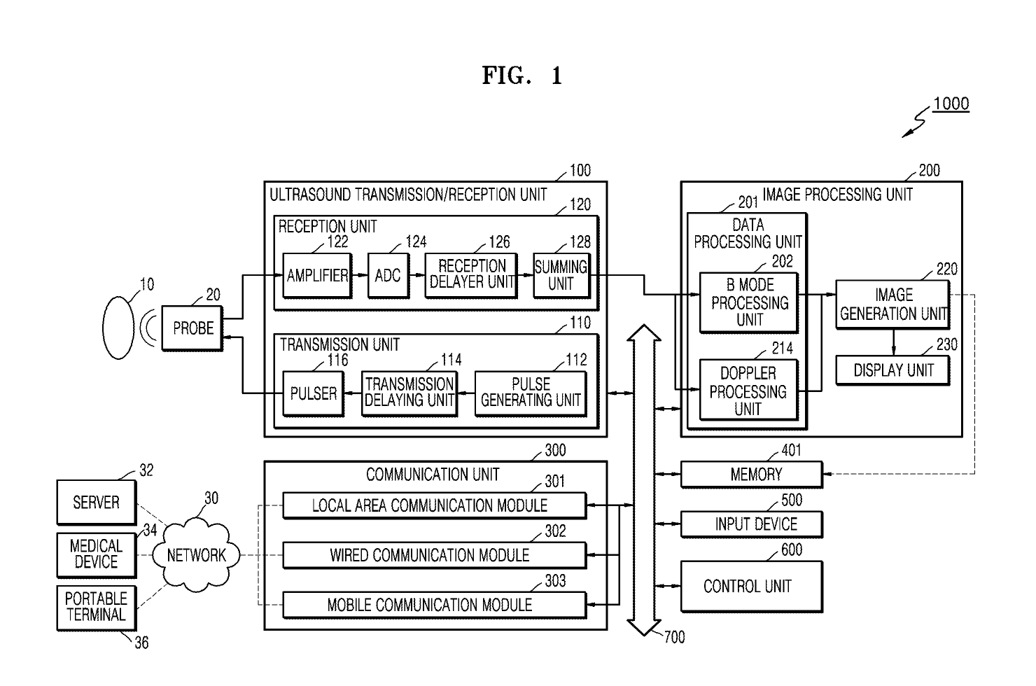

FIG. 1 is a block diagram illustrating a configuration of an ultrasound diagnosis apparatus 1000 according to an exemplary embodiment. Referring to FIG. 1, the ultrasound diagnosis apparatus 1000 may include a probe 20, an ultrasound transmission/reception unit 100, an image processing unit 200, a communication unit 300, a memory 401, an input device 500, and a control unit 600, which may be connected to one another via buses 700.

The ultrasound diagnosis apparatus 1000 may be a cart type apparatus or a portable type apparatus. Examples of portable ultrasound diagnosis apparatuses may include, but are not limited to, a picture archiving and communication system (PACS) viewer, a smartphone, a laptop computer, a personal digital assistant (PDA), and a tablet PC.

The probe 20 transmits ultrasound waves to an object 10 in response to a driving signal applied by the ultrasound transmission/reception unit 100 and receives echo signals reflected by the object 10. The probe 20 includes a plurality of transducers, and the plurality of transducers oscillate in response to electric signals and generate acoustic energy, that is, ultrasound waves. Furthermore, the probe 20 may be connected to the main body of the ultrasound diagnosis apparatus 1000 by wire or wirelessly, and according to some embodiments, the ultrasound diagnosis apparatus 1000 may include a plurality of probes 20.

A transmission unit 110 supplies a driving signal to the probe 20. The transmission unit 110 includes a pulse generating unit 112, a transmission delaying unit 114, and a pulser 116. The pulse generating unit 112 generates pulses for forming transmission ultrasound waves based on a predetermined pulse repetition frequency (PRF), and the transmission delaying unit 114 delays the pulses by delay times necessary for determining transmission directionality. The pulses which have been delayed correspond to a plurality of piezoelectric vibrators included in the probe 20, respectively. The pulser 116 applies a driving signal (or a driving pulse) to the probe 20 based on timing corresponding to each of the pulses which have been delayed.

A reception unit 120 generates ultrasound data by processing echo signals received from the probe 20. The reception unit 120 may include an amplifier 122, an analog-to-digital converter (ADC) 124, a reception delaying unit 126, and a summing unit 128. The amplifier 122 amplifies echo signals in each channel, and the ADC 124 performs analog-to-digital conversion with respect to the amplified echo signals. The reception delaying unit 126 delays digital echo signals output by the ADC 124 by delay times necessary for determining reception directionality, and the summing unit 128 generates ultrasound data by summing the echo signals processed by the reception delaying unit 166. In some embodiments, the reception unit 120 may not include the amplifier 122. In other words, if the sensitivity of the probe 20 or the capability of the ADC 124 to process bits is enhanced, the amplifier 122 may be omitted.

The image processing unit 200 generates an ultrasound image by scan-converting ultrasound data generated by the ultrasound transmission/reception unit 100 and displays the ultrasound image. The ultrasound image may be not only a grayscale ultrasound image acquired by scanning an object in an amplitude (A) mode, a brightness (B) mode, and a motion (M) mode, but also a Doppler image showing a movement of an object via a Doppler effect. The Doppler image may be a blood flow Doppler image showing flow of blood (also referred to as a color Doppler image), a tissue Doppler image showing a movement of tissue, or a spectral Doppler image showing a moving speed of an object as a waveform.

A B mode processing unit 202 extracts B mode components from ultrasound data and processes the B mode components. An image generating unit 220 may generate an ultrasound image indicating signal intensities as brightness based on the extracted B mode components.

Similarly, a Doppler processing unit 214 may extract Doppler components from ultrasound data, and the image generating unit 220 may generate a Doppler image indicating a movement of an object as colors or waveforms based on the extracted Doppler components.

According to an exemplary embodiment, the image generating unit 220 may generate a three-dimensional (3D) ultrasound image via volume-rendering with respect to volume data and may also generate an elasticity image by imaging deformation of the object 10 due to pressure. Furthermore, the image generating unit 220 may display various pieces of additional information in an ultrasound image by using text and graphics. In addition, the generated ultrasound image may be stored in the memory 401.

A display unit 230 displays the generated ultrasound image. The display unit 230 may display not only an ultrasound image, but also various pieces of information processed by the ultrasound diagnosis apparatus 1000 on a screen image via a graphical user interface (GUI). In addition, the ultrasound diagnosis apparatus 1000 may include two or more displays 230 according to some embodiments.

The communication unit 300 is connected to a network 30 by wire or wirelessly to communicate with an external device or a server. The communication unit 300 may exchange data with a hospital server or another medical apparatus in a hospital, which is connected thereto via a PACS. Furthermore, the communication unit 300 may perform data communication according to the digital imaging and communications in medicine (DICOM) standard.

The communication unit 300 may transmit or receive data related to diagnosis of an object, e.g., an ultrasound image, ultrasound data, and Doppler data of the object, via the network 30 and may also transmit or receive medical images captured by another medical apparatus, e.g., a computed tomography (CT) apparatus, a magnetic resonance imaging (MRI) apparatus, or an X-ray apparatus. Furthermore, the communication unit 300 may receive information about a diagnosis history or medical treatment schedule of a patient from a server and utilizes the received information to diagnose the patient. Furthermore, the communication unit 300 may perform data communication not only with a server or a medical apparatus in a hospital, but also with a portable terminal of a medical doctor or patient.

The communication unit 300 is connected to the network 30 by wire or wirelessly to exchange data with a server 32, a medical apparatus 34, or a portable terminal 36. The communication unit 300 may include one or more components for communication with external devices. For example, the communication unit 300 may include a local area communication module 301, a wired communication module 302, and a mobile communication module 303.

The local area communication module 301 refers to a module for local area communication within a predetermined distance. Examples of local area communication techniques according to an exemplary embodiment may include, but are not limited to, wireless LAN, Wi-Fi, Bluetooth, ZigBee, Wi-Fi Direct (WFD), ultra wideband (UWB), infrared data association (IrDA), Bluetooth low energy (BLE), and near field communication (NFC).

The wired communication module 302 refers to a module for communication using electric signals or optical signals. Examples of wired communication techniques according to an exemplary embodiment may include communication via a twisted pair cable, a coaxial cable, an optical fiber cable, and an Ethernet cable.

The mobile communication module 303 transmits or receives wireless signals to or from at least one selected from a base station, an external terminal, and a server on a mobile communication network. The wireless signals may be voice call signals, video call signals, or various types of data for transmission and reception of text/multimedia messages.

The memory 401 stores various data processed by the ultrasound diagnosis apparatus 1000. For example, the memory 401 may store medical data related to diagnosis of an object, such as ultrasound data and an ultrasound image that are input or output, and may also store algorithms or programs which are to be executed in the ultrasound diagnosis apparatus 1000.

The memory 401 may be any of various storage media, e.g., a flash memory, a hard disk drive, EEPROM, etc. Furthermore, the ultrasound diagnosis apparatus 1000 may utilize web storage or a cloud server that performs the storage function of the memory 401 online.

The input device 500 refers to a means via which a user inputs data for controlling the ultrasound diagnosis apparatus 1000. The input device 500 may include hardware components, such as a keypad, a mouse, a touch panel, a touch screen, and a jog switch. However, exemplary embodiments are not limited thereto, and the input device 500 may further include any of various other input units including an electrocardiogram (ECG) measuring module, a respiration measuring module, a voice recognition sensor, a gesture recognition sensor, a fingerprint recognition sensor, an iris recognition sensor, a depth sensor, a distance sensor, etc.

The control unit 600 may control all operations of the ultrasound diagnosis apparatus 1000. In other words, the control unit 600 may control operations among the probe 20, the ultrasound transmission/reception unit 100, the image processing unit 200, the communication unit 300, the memory 401, and the input device 500 illustrated in FIG. 1.

All or some of the probe 20, the ultrasound transmission/reception unit 100, the image processing unit 200, the communication unit 300, the memory 401, the input device 500, and the control unit 600 may be implemented as software modules. However, exemplary embodiments are not limited thereto, and some of the components stated above may be implemented as hardware modules. Furthermore, at least one selected from the ultrasound transmission/reception unit 100, the image processing unit 200, and the communication unit 300 may be included in the control unit 600. However, exemplary embodiments are not limited thereto.

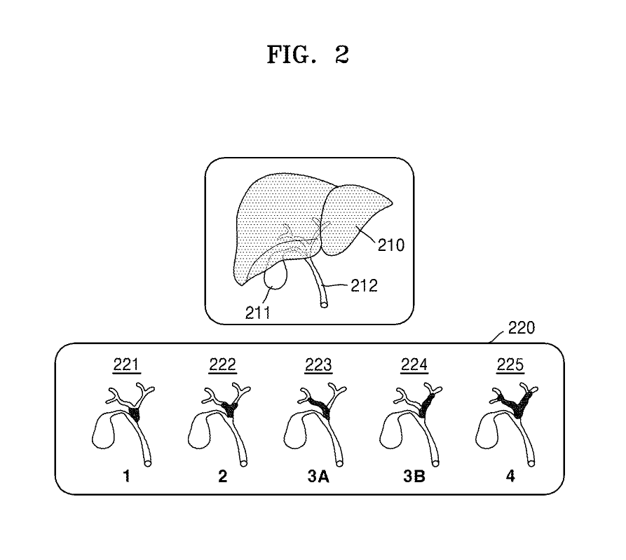

FIG. 2 illustrates types of bile duct tumors. A gallbladder 211 is a branch-shaped pouch that concentrates and stores bile produced in a liver 210. The gallbladder 211 helps fat digestion by secreting the bile into the duodenum through a bile duct 212. If a tumor occurs in the bile duct 212, since the bile duct 212 may be blocked by the tumor, a resection operation may be performed to remove the tumor. In order to perform the resection operation, it is important to detect a tumor shape. In general, the tumor shape may be classified into five types. For example, the tumor shape may be classified into a tumor type I 221, a tumor type II 222, a tumor type IIIA 223, a tumor type IIIB 224, and a tumor type IV 225. It is important to detect the tumor shape in order to determine a resection pattern of the bile duct 212.

Referring to FIG. 2, if the tumor is of the tumor type I 221, the tumor is restricted within a common hepatic duct under a convergence point of a right hepatic duct and a left hepatic duct. If the tumor is of the tumor type II 222, the tumor includes the convergence point of the right hepatic duct and the left hepatic duct. If the tumor is of the tumor type IIIA 223, the tumor extends to a divergence point of the right hepatic duct in addition to the tumor type II 222. If the tumor is of the tumor type IIIB 224, the tumor extends to a divergence point of the left hepatic duct in addition to the tumor type II 222. If the tumor is of the tumor type IV 225, the tumor extends to both the divergence point of the right hepatic duct and the divergence point of the left hepatic duct.

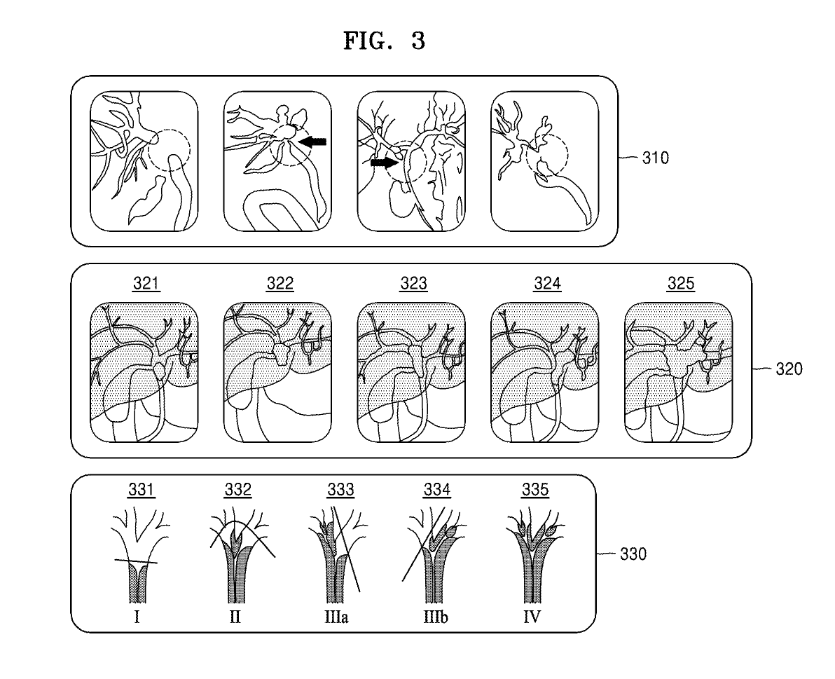

FIG. 3 illustrates computerized tomography (CT) images 310, rendered images 320, and resection pattern images 330 of a bile duct and a gallbladder.

In the related art, images like the CT images 310 of the bile duct are captured in order to detect a tumor distribution, and a medical doctor determines a tumor type on the basis of the captured CT images 310 and a resection pattern for removing the tumor on the basis of the determined tumor type.

For example, the CT images 310 are an array of captured CT images of the bile duct. A user has to determine a tumor position from the CT images 310. However, capturing CT images is time-consuming and determining the tumor position may vary with the user.

In order to prevent such problems, in some exemplary embodiments, ultrasound images of an object may be captured and then converted into 3D-rendered images 321 to 325 in real time, and resection patterns 331 to 335 for tumor removal may be automatically determined by an ultrasound diagnosis apparatus on the basis of the 3D-rendered images 321 to 325.

The 3D-rendered images 321 to 325 illustrated in FIG. 3 are examples of 3D-rendered images of the CT images 310. The tumor position may be clearly detected from the 3D-rendered images 321 to 325.

Although the ultrasound diagnosis apparatus according to an exemplary embodiment generates the 3D-rendered images from the ultrasound images, the CT images 310 are illustrated for reference.

The resection pattern images 330 represent the resection patterns 331 to 335 desirable according to the current statuses of the tumor. For example, when the tumor corresponds to a tumor type I, the resection pattern 331 may be selected for removal of the current tumor. Likewise, when the tumor corresponds to a tumor type II/IIIa/IIIb/IV, the resection pattern 332/333/334/335 may be selected for removal of the current tumor.

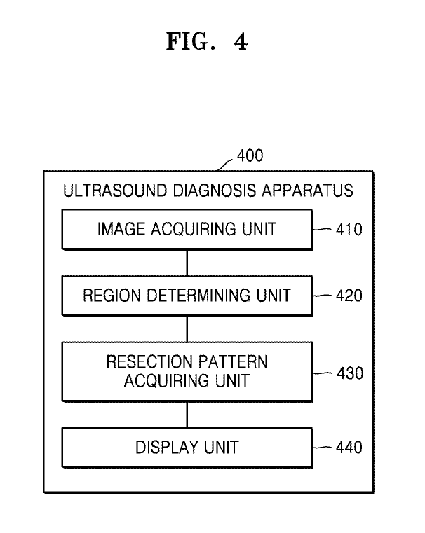

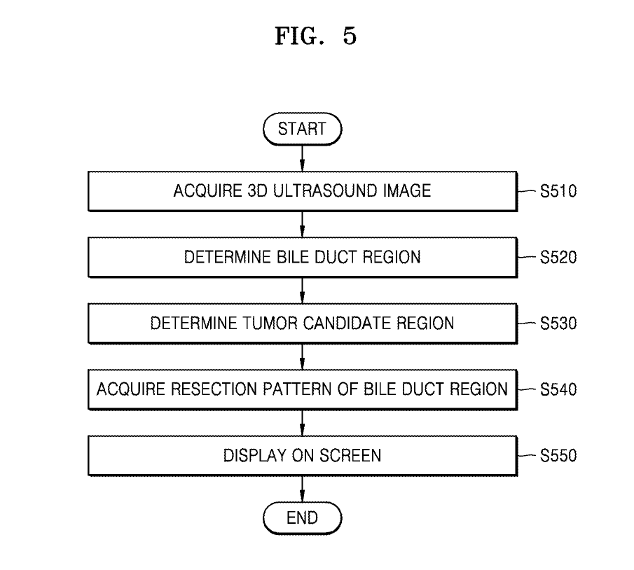

A method of determining the resection pattern will be described in detail with reference to FIGS. 4 and 5.

FIG. 4 is a block diagram of an ultrasound diagnosis apparatus according to some exemplary embodiments.

Referring to FIG. 4, an ultrasound diagnosis apparatus 400 according to some exemplary embodiments may include an image acquiring unit 410, a region determining unit 420, a resection pattern acquiring unit 430, and a display unit 440. Although the image acquiring unit 410, the region determining unit 420, and the resection pattern acquiring unit 430 are illustrated separately in FIG. 4, exemplary embodiments are not limited thereto and they may be implemented by one processor.

The ultrasound diagnosis apparatus 400 may perform some or all of the functions performed by the ultrasound diagnosis apparatus 1000.

The image acquiring unit 410 acquires a 3D ultrasound image of a partial region of an object. The region determining unit 420 determines a bile duct region and a tumor candidate region in the 3D ultrasound image. The resection pattern acquiring unit 430 acquires a resection pattern of the bile duct region by comparing a shape of the tumor candidate region and the bile duct region with a predetermined pattern. The display unit 440 displays on a screen the resection pattern of the bile duct region, which is acquired by the resection pattern acquiring unit 430.

The object may be a human or an animal, and the partial region of the object may be an anatomic part (e.g., a gallbladder, a bile duck, or the like) of the human or the animal. Also, the object may be a region including a gallbladder and a bile duct. In the present specification, the gallbladder region may be a region that is determined or labeled as a gallbladder in the 3D ultrasound image acquired by the apparatus. Also, the bile duct region may be a region that is determined or labeled as a bile duct in the 3D ultrasound image. The tumor candidate region may be a region that is determined or labeled as a tumor in the 3D ultrasound image by the ultrasound diagnosis apparatus. In the present specification, labeling may mean that the same label is attached to adjacent pixels included in the same region and different labels are attached to adjacent pixels included in different regions. Also, when a region (i.e., an image pixel set) is labeled, it may mean that the region is designated as an anatomic part by the ultrasound diagnosis apparatus.

FIG. 5 is a flow diagram illustrating an ultrasound diagnosis method according to some exemplary embodiments. In operation S510, the ultrasound diagnosis apparatus 400 acquires a 3D ultrasound image of an object.

In operation S520, the ultrasound diagnosis apparatus 400 determines a bile duct region.

In order to determine the bile duct region, the ultrasound diagnosis apparatus 400 may first determine a gallbladder region in the acquired 3D ultrasound image. In order to determine the gallbladder region, the ultrasound diagnosis apparatus 400 may detect a plurality of dark regions in the 3D ultrasound image. For example, the ultrasound diagnosis apparatus 400 may detect a set of pixels having a pixel brightness value less than or equal to a threshold value and determine the largest circular region in the detected pixel set as the gallbladder region. In detail, the ultrasound diagnosis apparatus 400 may acquire gallbladder candidate regions by comparing the detected pixel set with a predetermined pattern, for example, a predetermined pattern similar to a circular shape. Thereafter, the ultrasound diagnosis apparatus 400 may determine the largest of the gallbladder candidate regions as the gallbladder region. The above determination method is merely exemplary, and exemplary embodiments are not limited thereto. The ultrasound diagnosis apparatus 400 according to some exemplary embodiments may determine a region having a similar shape to a gallbladder as the gallbladder region in various ways. For example, the ultrasound diagnosis apparatus 400 may determine the gallbladder region via a machine learning-based algorithm. The determined gallbladder region may be labeled as the gallbladder region in the 3D ultrasound image.

After determining the gallbladder region, the ultrasound diagnosis apparatus 400 may detect a set of pixels that are adjacent to the gallbladder region and have a pixel brightness value less than or equal to a threshold value and also determine the largest duct-shaped region in the detected pixel set as the bile duct region. The largest duct-shaped region may be detected by selecting duct-shaped regions and comparing them with a predetermined pattern, and the region having the most similar shape to the bile duct region may be detected and determined as the bile duct region by the machine learning algorithm. Also, the determined bile duct region may be labeled as the bile duct region in the 3D ultrasound image.

In operation S530, the ultrasound diagnosis apparatus 400 determines a tumor candidate region. The tumor candidate region may be determined on the basis of the bile duct region. For example, the tumor candidate region may be determined on the basis of at least one of a Doppler signal of the bile duct region and a shape of the bile duct region.

If no tumor exists in the bile duct region, no Doppler signal is generated. If a tumor exists in the bile duct region, a Doppler signal is detected in the bile duct region. Depending on the tumor shape, a Doppler signal may appear as a strong line, or a plurality of Doppler signals may appear as weak lines. The tumor candidate region may be determined on the basis of the Doppler signal.

Also, the bile duct shape may vary according to the tumor shape. For example, when the cross-sectional shape of the bile duct is distorted, it may be estimated that a tumor exists therein.

Thus, the ultrasound diagnosis apparatus 400 may determine the tumor candidate region by comparison with a predetermined pattern in consideration of the Doppler signal of the bile duct region or the cross-sectional shape of the bile duct. Also, the tumor candidate region may be determined by additionally comparing a tumor possibility score based on the cross-sectional shape thereof, the existence/nonexistence thereof, and the strength of the Doppler signal.

In operation S540, the ultrasound diagnosis apparatus 400 may acquire a resection pattern of the bile duct region by comparing a shape of the determined tumor candidate region and the determined bile duct region with a predetermined pattern. The resection pattern of the bile duct region may correspond to any one of the resection patterns 331 to 335 illustrated in FIG. 3.

In detail, the ultrasound diagnosis apparatus 400 detects the most similar pattern by comparing the shape of the determined tumor candidate region and the determined bile duct region with the tumor types I, II, IIIa, IIIb, and IV illustrated in FIG. 3. For example, when it is determined that the most similar pattern corresponds to the tumor type I, the resection pattern of the bile duct may be determined as the resection pattern 331.

In operation S550, the ultrasound diagnosis apparatus 400 displays on the screen a resection pattern image including the determined resection pattern. The resection pattern image may correspond to one of the resection patterns 331 to 335 illustrated in FIG. 3.

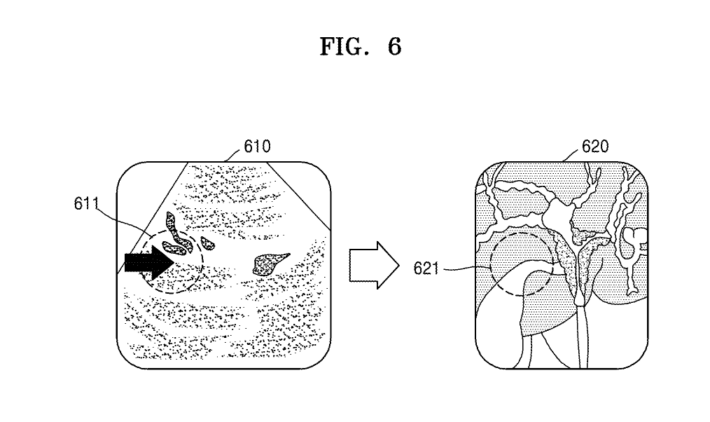

FIG. 6 illustrates a tumor display screen of the ultrasound diagnosis apparatus 400 according to some exemplary embodiments.

FIG. 6 illustrates an ultrasound image 620 of the bile duct region and a 3D-rendered image 610 of the bile duct region. The ultrasound diagnosis apparatus 400 may acquire the 3D-rendered image 610 of the bile duct region from the ultrasound image 620 of the bile duct region. A tumor candidate region 621 of the ultrasound image 620 may correspond to a tumor candidate region 611 of the 3D-rendered image 610 of the bile duct region.

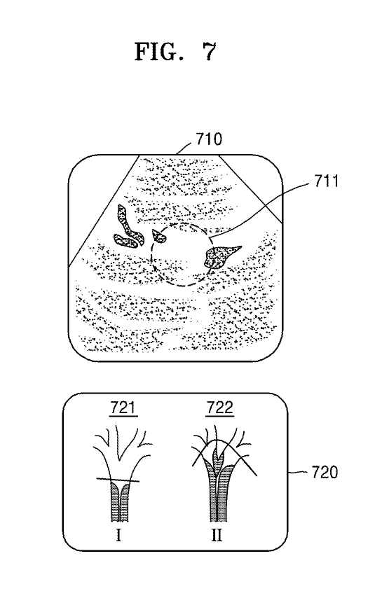

FIG. 7 illustrates a screen displaying a resection pattern image 720 based on a determined tumor candidate region 711 according to some exemplary embodiments.

A 3D ultrasound image 710 of the bile duct region may include the tumor candidate region 711. A resection pattern may be determined according to the shape of the tumor candidate region 711, and the determined resection pattern may appear as a determined resection pattern I 721 or a determined resection pattern II 722 on the screen.

Although only two determined resection patterns are illustrated in the resection pattern image 720, only one resection pattern may be determined and illustrated therein according to the tumor shape. According to other exemplary embodiments, three or four resection patterns may be determined and illustrated therein. When a plurality of resection patterns are determined, the desirable order of the resection patterns may be displayed (not illustrated). For example, the most desirable resection pattern may be illustrated as “first-priority resection pattern”, and the second most desirable resection pattern may be illustrated as “second-priority resection pattern”, thus allowing the user to conveniently identify the most desirable resection pattern.

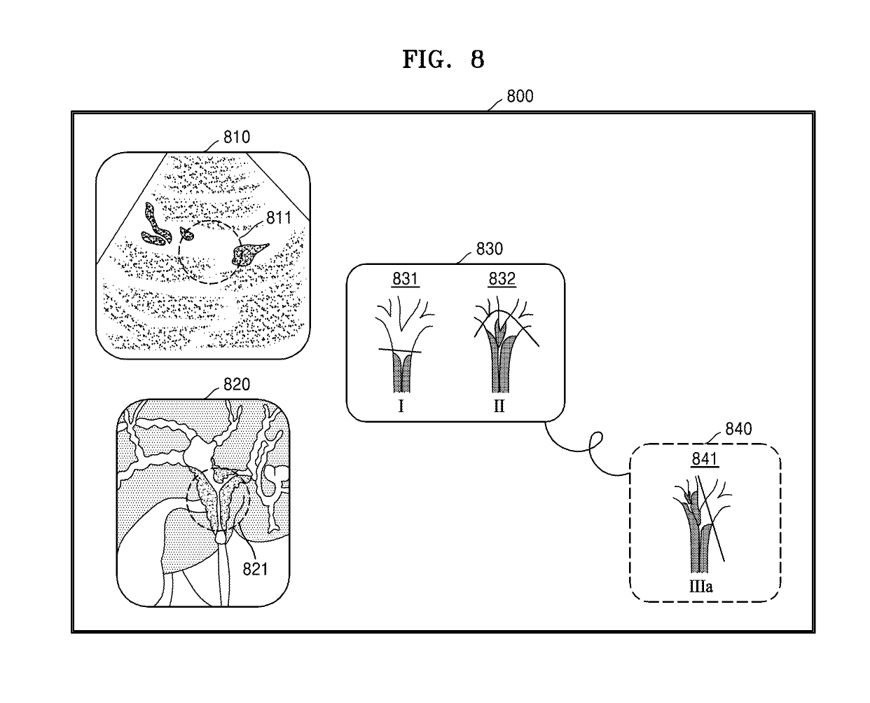

FIG. 8 is a diagram illustrating an example of displaying an ultrasound image 810, an auxiliary image 820, and a resection pattern image 830 on a screen 800 according to some exemplary embodiments.

The auxiliary image 820 may be a 3D-rendered image of the ultrasound image 810.

According to some exemplary embodiments, the ultrasound image 810 may be displayed at a left top of the screen 800, and the auxiliary image 820 may be displayed at a left bottom of the screen 800. Also, the resection pattern image 830 determined on the basis of a tumor candidate region may be displayed at a right side of the screen 800. However, such a display layout is merely exemplary and exemplary embodiments are not limited thereto.

The display unit 440 may display a bile duct region and a gallbladder region of the auxiliary image 820 in different colors and may also display blood vessels around the bile duct region or the gallbladder region in different colors. Also, the display unit 440 may display tumor candidate regions according to various types of indicators. An arrow in FIG. 6 or a circular dotted line in FIGS. 6, 7, and 8 is a type of indicator. Indicators of tumor candidate regions may be displayed in various modes such as colors and figures. Also, the tumor candidate region of the auxiliary image 820 may be displayed in a different color in the bile duct region of the auxiliary image 820. For example, when the bile duct region is displayed in yellow, the tumor candidate region may be displayed in red in the bile duct region.

When the user modifies a tumor candidate region 811 in the ultrasound image 810 or a tumor candidate region 821 in the auxiliary image 820, the ultrasound diagnosis apparatus 400 may modify the resection pattern image 830 and display a modified resection pattern image 840. For example, when a resection pattern image I 831 and a resection pattern image II 832 are displayed as the resection patterns determined according to the existing tumor candidate regions, only a resection pattern image IIIa based on the modified tumor candidate region may be displayed.

That is, the user may examine the tumor candidate region, which is automatically determined by the ultrasound diagnosis apparatus 400, and acquire a proper resection pattern image by correcting any incorrect portion thereof.

It should be understood that the exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope of the inventive concept as defined by the following claims.

What is claimed is:

1. An ultrasound diagnosis apparatus, comprising:
an image acquiring unit configured to acquire a three-dimensional (3D) ultrasound image of a partial region of an object;
a region determining unit configured to determine a bile duct region and a tumor candidate region in the 3D ultrasound image;
a resection pattern acquiring unit configured to acquire a resection pattern of the bile duct region by comparing a shape of the tumor candidate region and the bile duct region with a predetermined pattern; and
a display unit configured to display on a screen a resection pattern image including the resection pattern of the bile duct region.

2. The ultrasound diagnosis apparatus of claim 1, wherein the region determining unit determines gallbladder candidate regions by comparing regions in the 3D ultrasound image which have a pixel brightness value less than or equal to a threshold value with a predetermined shape, and determines the largest of the gallbladder candidate regions as a gallbladder region by comparing sizes of the gallbladder candidate regions.

3. The ultrasound diagnosis apparatus of claim 2, wherein the region determining unit determines bile duct candidate regions by comparing regions which are adjacent to the gallbladder region and have a pixel brightness value less than or equal to a threshold value with a predetermined shape, and determines the largest of the bile duct candidate regions as the bile duct region by comparing sizes of the bile duct candidate regions.

4. The ultrasound diagnosis apparatus of claim 1, wherein the region determining unit determines the tumor candidate region on the basis of at least one of a Doppler signal of the bile duct region and a shape of the bile duct region.

5. The ultrasound diagnosis apparatus of claim 1, wherein when the tumor candidate region displayed on the screen is modified by an external input, the resection pattern acquiring unit modifies the resection pattern on the basis of the modified tumor candidate region and the display unit displays the modified resection pattern on the screen.

6. The ultrasound diagnosis apparatus of claim 2, wherein the display unit displays on the screen an ultrasound image including at least one of the gallbladder region, the bile duct region, and the tumor candidate region.

7. The ultrasound diagnosis apparatus of claim 6, wherein the display unit displays an auxiliary image on the screen, wherein the auxiliary image is a 3D-rendered image of the ultrasound image.

8. The ultrasound diagnosis apparatus of claim 7, wherein the display unit displays at least one of the resection pattern image, the ultrasound image, and the auxiliary image on the screen.

9. The ultrasound diagnosis apparatus of claim 7, wherein the display unit displays a blood vessel, the bile duct region, and the gallbladder region of the auxiliary image in different colors.

10. The ultrasound diagnosis apparatus of claim 7, wherein the display unit displays the tumor candidate region of the auxiliary image in a different color in the bile duct region.

11. The ultrasound diagnosis apparatus of claim 1, wherein the display unit displays the tumor candidate region according to at least one type of indicator.

12. An ultrasound diagnosis method, comprising:
acquiring a three-dimensional (3D) ultrasound image of a partial region of an object;
determining a bile duct region and a tumor candidate region in the 3D ultrasound image;
acquiring a resection pattern of the bile duct region by comparing a shape of the tumor candidate region and the bile duct region with a predetermined pattern; and
displaying on a screen a resection pattern image including the resection pattern of the bile duct region.

13. The ultrasound diagnosis method of claim 12, wherein the determining of the bile duct region and the tumor candidate region comprises:
determining gallbladder candidate regions by comparing regions in the 3D ultrasound image which have a pixel brightness value less than or equal to a threshold value with a predetermined shape; and
determining the largest of the gallbladder candidate regions as a gallbladder region by comparing sizes of the gallbladder candidate regions.

14. The ultrasound diagnosis method of claim 13, wherein the determining of the bile duct region and the tumor candidate region further comprises:
determining bile duct candidate regions by comparing regions which are adjacent to the gallbladder region and have a pixel brightness value less than or equal to a threshold value with a predetermined shape; and
determining the largest of the bile duct candidate regions as the bile duct region by comparing sizes of the bile duct candidate regions.

15. The ultrasound diagnosis method of claim 12, wherein the determining of the bile duct region and the tumor candidate region comprises determining the tumor candidate region on the basis of at least one of a Doppler signal of the bile duct region and a shape of the bile duct region.

16. The ultrasound diagnosis method of claim 12, wherein when the tumor candidate region displayed on the screen is modified by an external input, the resection pattern is modified on the basis of the modified tumor candidate region, and the modified resection pattern is displayed on the screen.

17. The ultrasound diagnosis method of claim 13, wherein the displaying of the resection pattern image including the resection pattern of the bile duct region on the screen comprises displaying on the screen an ultrasound image including at least one of the gallbladder region, the bile duct region, and the tumor candidate region.

18. The ultrasound diagnosis method of claim 17, wherein the displaying of the resection pattern image including the resection pattern of the bile duct region on the screen further comprises displaying an auxiliary image on the screen, wherein the auxiliary image is a 3D-rendered image of the ultrasound image.

19. The ultrasound diagnosis method of claim 18, wherein the displaying of the resection pattern image including the resection pattern of the bile duct region on the screen further comprises displaying at least one of the resection pattern image, the ultrasound image, and the auxiliary image on the screen.

20. The ultrasound diagnosis method of claim 18, wherein the displaying of the resection pattern image including the resection pattern of the bile duct region on the screen further comprises displaying a blood vessel, the bile duct region, and the gallbladder region of the auxiliary image in different colors.

21. The ultrasound diagnosis method of claim 18, wherein the displaying of the resection pattern image including the resection pattern of the bile duct region on the screen further comprises displaying the tumor candidate region of the auxiliary image in a different color in the bile duct region.

22. The ultrasound diagnosis method of claim 12, wherein the displaying of the resection pattern image including the resection pattern of the bile duct region on the screen comprises displaying the tumor candidate region according to at least one type of indicator.

23. A non-transitory computer-readable recording medium that stores a program that performs the ultrasound diagnosis method of claim 12 when executed by a computer.
